(19) United States
(12) Patent Application Publication
(10) Pub. No.: US 2014/0037049 A1
(43) Pub. Date: Feb. 6, 2014

(54) SYSTEMS AND METHODS FOR INTERVENTIONAL IMAGING

(71) Applicants: David Allen Langan, Clifton Park, NY (US); Peter Michael Edic, Albany, NY (US); Bernhard Erich Hermann Claus, Niskayuna, NY (US)

(72) Inventors: David Allen Langan, Clifton Park, NY (US); Peter Michael Edic, Albany, NY (US); Bernhard Erich Hermann Claus, Niskayuna, NY (US)

(73) Assignee: GENERAL ELECTRIC COMPANY, SCHENECTADY, NY (US)

(21) Appl. No.: 13/563,731

(22) Filed: Jul. 31, 2012

(51) Int. Cl. A61B 6/03

(52) U.S. Cl. CPC A61B 6/032
USPC 378/20; 378/4

(57) ABSTRACT

An imaging system including an integral computed tomography and interventional (CT/I) system that includes a large-area detector configured to acquire projection data corresponding to a field of view of the system from one or more view angles is presented. The system includes a computing device operatively coupled to the CT/I system and configured to process the acquired projection data to generate a 2D projection image in real-time, a 3D cross-sectional image of a region of interest in the subject, a combined image using the 2D projection image and the 3D cross-sectional image and/or control selective generation of the 2D projection image, the 3D cross-sectional image and/or the combined image based on one or more imaging specifications. The system also includes a display operatively coupled to the computing device and configured to display the 2D projection image, the 3D cross-sectional image and/or the combined image based on the imaging specifications.

### BACKGROUND

Embodiments of the present disclosure relate generally to interventional imaging, and more particularly to integral CT and interventional systems and methods for seamless diagnostic and interventional imaging.

Interventional techniques are widely used for managing a plurality of life-threatening medical conditions. Particularly, certain interventional techniques entail minimally invasive image-guided procedures that provide a cost-effective alternative to invasive surgery. Interventional imaging, for example, may be employed for diagnosing and treating patients who may be suffering from heart disease, coronary artery disease, stroke, osteoporosis, cancer and other medical conditions. In such patients, interventional radiology may facilitate minimally-invasive procedures without the stress of a surgical operation.

Generally, interventional techniques may be employed in various fields of medicine such as neurology, general radiology, cardiology and electrophysiology. Particularly, interventional techniques may be used in a plurality of medical diagnostic and therapeutic procedures. These procedures, for example, include tissue anomaly detection, angioplasty, stent placement, coil placement, thrombolysis, embolysis, and heart valve replacement. Specifically, interventional imaging systems may be used to visualize segments of a vascular system that may be difficult to visualize using other imaging techniques due to obstructions and/or cardiac and respiratory movements. In a patient with cardiac disease, for example, a suitable interventional procedure may be employed to provide therapy at the site of a stenosis of a vessel. Similarly, interventional techniques may be used to detect and dissolve a blood clot inside the skull using fibrinolytic agents.

Certain medical procedures may require functional information in addition to anatomical information for facilitating the interventional procedure. Knowledge of functional information such as tissue perfusion parameters including regional blood volume, regional mean transit time, regional blood flow and permeability surface area are very useful in various medical scenarios. A surgeon may rely on these functional parameters before planning the surgical procedure during endovascular treatments, for example, concerning cerebral vascular accidents, angioplasties of the carotid and placement of carotidian and intracranial stents. The surgeon may also use the functional parameters during the interventional procedure for evaluating the efficacy of the therapeutic procedure in real-time, and further for determining whether to stop or continue the procedure based on the evaluated effect.

Generally, a patient is initially examined by a magnetic resonance (MR) system or a computed tomography (CT) system to obtain both anatomical and functional information for diagnosis and/or treatment. Subsequently, the patient undergoes therapy via an interventional procedure in a vascular operating theater. During the operation, an interventional device such as a catheter may be inserted into a vascular structure, allowing access to a region of interest (ROI), such as the thoracic cavity for performing the interventional procedure. The insertion as well as the navigation of the catheter within the different branches of the vascular system, however, is a challenging procedure.

Conventionally, the navigation of the catheter through the vasculature of the patient 104, as shown in FIG. 1, is guided fluoroscopically. Accordingly, a transient bolus of contrast agent is administered via the catheter for allowing a two-dimensional (2D) projection of blood vessels in the vicinity of the catheter tip using x-ray exposures. Conventional fluoroscopic interventional systems, however, may constrain a medical practitioner's understanding of a relation between the catheter and vascular positions primarily based on 2D projection images that provide limited or no information in the depth direction, where the depth direction is parallel to the x-rays that traverse the patient. Further, certain fluoroscopic procedures may entail frequent switching between aligning the patient along a desired imaging plane and navigating the catheter inside the patient's body. Consequently, lack of sufficient information in fluoroscopic interventional imaging can result in longer examination time, which may result in increased ionizing radiation dose and exhaustion to the medical practitioner, and increased ionizing radiation dose, contrast dose, anesthesia time, and discomfort to the patient. Additionally, risk of injury to the patient increases with the scanning duration and/or retention of a catheter in the patient's vasculature.

Generally, it may be desirable to acquire both 2D projection imaging data, as well as three-dimensional (3D) cross-sectional imaging data using CT, while performing certain diagnostic and therapeutic procedures. Particularly, in interventional procedures, use of the 2D projection data having high temporal sampling, high-spatial resolution, and lower latency between data collection and image presentation is of great significance for real-time device guidance and therapeutic delivery. However, 2D projection imaging data may prove inadequate in certain scenarios due to confounding information (overlaying structures) in the acquired projection information. Use of the 3D cross-sectional imaging data may allow both anatomical and functional assessments of an outcome of the interventional procedure. However, slower rotation speeds achieved with conventional interventional systems may result in image artifacts in 3D imaging due to voluntary motion, such as due to patient repositioning, and involuntary motion such as peristalsis or heart motion within the patient.

Additionally, certain interventional systems may employ detectors with smaller axial coverage when compared to conventional CT systems, which may cause a truncated imaging field of view (FOV), leading to erroneous measurements and additional scanning time. The erroneous measurements and the long examination times may prove detrimental to patient health especially in emergencies such as assessment of ischemia in the brain and heart, where immediate and accurate assessment from interactive evaluation of real-time images is critical for improving patient health.

Certain conventional fluoroscopic interventional systems implement bi-planar operation to provide limited 3D information at high temporal sampling, for example of about 33 frames per second, and efficient dose utilization. Other systems, for example C-arm systems, may employ computed tomography and/or tomosynthesis for reconstructing 3D representations from measured projection data. However, conventional C-arm systems rotate too slowly to generate dynamic 3D information during medical procedures such as neurological imaging, during which it is desirable to reconstruct multiple 3D images to characterize passage of a bolus in real-time. Lack of dynamic 3D data during imaging results in uncertainty, which in turn may lead to incorrect diagnoses, treatment planning, and/or procedure validation.

Thus, neither conventional fluoroscopic interventional systems, nor C-arm systems allow for acquisition of both high-fidelity 2D projection images and 3D cross-section images of the entire organ using the same system to conduct diagnostic evaluation as well as perform interventional procedures.

### BRIEF DESCRIPTION

In accordance with aspects of the present disclosure, an imaging system is presented. An imaging system is presented. The imaging system an integral computed tomography and interventional system including a large-area detector configured to acquire projection data corresponding to a field of view of the imaging system from one or more view angles, The imaging system also includes a computing device operatively coupled to the integral computed tomography and interventional system, where the computing device is configured to perform one or more of process the projection data acquired by the large-area detector to generate a two-dimensional projection image in real-time, process the projection data acquired by the large-area detector to generate a three-dimensional cross-sectional image of a region of interest in the subject, generate a combined image using the two-dimensional projection image and the three-dimensional cross-sectional image, and control selective generation of the two-dimensional projection image, the three-dimensional cross-sectional image, the combined image, or combinations thereof, based on one or more imaging specifications. Furthermore, the imaging system also includes a display device operatively coupled to the computing device and configured to display one or more of the two-dimensional projection image, the three-dimensional cross-sectional image, the combined image, or combinations thereof, based on the one or more imaging specifications.

In accordance with another aspect of the present disclosure, an imaging method is presented. The method includes acquiring projection data corresponding to a region of interest from one or more view angles using a large-area detector in an integral computed tomography and interventional imaging system. Furthermore, the method includes processing the projection data to generate one or more of a two-dimensional projection image, a three-dimensional cross-sectional image of the region of interest, a combined image generated using the two-dimensional projection image and the three-dimensional cross-sectional image, or combinations thereof. Additionally, the method includes selectively displaying the two-dimensional projection image, the three-dimensional cross-sectional image, the combined image, or combinations thereof, in real-time based on one or more imaging specifications.

In accordance with certain aspects of the present disclosure, a non-transitory computer readable medium that stores instructions executable by one or more processors to perform a method for imaging is presented. Particularly, the non-transitory computer readable medium includes instructions for acquiring projection data corresponding to a region of interest from one or more view angles using a large-area detector in an integral computed tomography and interventional imaging system. Furthermore, the instructions allow for processing of the projection data to generate one or more of a two-dimensional projection image, a three-dimensional cross-sectional image of the region of interest, a combined image generated using the two-dimensional projection image and the three-dimensional cross-sectional image, or combinations thereof. Additionally, the instructions allow selectively displaying the two-dimensional projection image, the three-dimensional cross-sectional image, the combined image, or combinations thereof, in real-time based on one or more imaging specifications.

### DRAWINGS

These and other features and aspects of embodiments of the present technique will become better understood when the following detailed description is read with reference to the accompanying drawings in which like characters represent like parts throughout the drawings, wherein:

FIG. 1 is a schematic representation of an exemplary imaging system, in accordance with certain aspects of the present disclosure;

FIG. 2 is a schematic representation of an exemplary embodiment of an integral CT/Interventional (CT/I) system, in accordance with certain other aspects of the present disclosure; and

FIG. 3 is a flow diagram illustrating an exemplary method for CT/I assisted interventional imaging, in accordance with certain aspects of the present disclosure.

### DETAILED DESCRIPTION

The following description presents embodiments of imaging systems and methods that provide high-fidelity 2D projection and 3D volumetric images of a scanned organ for use in diagnostic and interventional procedures using reduced radiation dosage and examination time. The interventional procedures, for example, may include angioplasty, stent placement, balloon septostomy, Transcatheter Aortic-Valve Implantation (TAVI), localized thrombolytic drug administration, tumor embolization and/or an electrophysiology study.

Additionally, the following description presents embodiments of imaging systems and methods that minimize contrast agent dosage, x-ray radiation exposure and scan durations. Certain embodiments of the present systems and methods may also be used for reconstructing high-fidelity 3D cross-sectional images in addition to the 2D projection images for allowing real-time diagnosis and/or therapy delivery, and efficacy assessment. Particularly, certain embodiments illustrated herein describe an integral CT and interventional (CT/I) system that provides functionality not provided separately by either a CT or an interventional system. The CT/I system, for example, allows implementation of efficient protocols for neuro-perfusion assessment, while also providing real-time 2D imaging needed for device guidance and therapy delivery.

In one embodiment the integral CT/I system comprises an enclosed gantry supporting at least one x-ray source and detector in which the gantry is capable of rotation speeds of more than one rotation per second. This capability supports the collection of x-ray projection data to be used in the formation of 3D volumetric images either from a full scan (360 degrees), a half scan (180 degree plus fan angle), or other angular ranges (e.g., limited angle tomosynthesis, or stereo and other multi-view configurations), where each 3D volumetric image represents high-fidelity data acquired over a short time window. In one embodiment, one or more such 3D volumes enable the evaluation of functional information of the imaged region of interest. In another embodiment, the gantry can be positioned at a specific angular orientation, tilt angle, and position, and a 2D projection image (or a sequence of 2D projection images) can be acquired, for example, in order to enable imaging during an interventional procedure. For example, such a system may be used to perform a diagnostic scan (resulting in a 3D volumetric image), provide imaging during an interventional procedure (as a sequence of 2D projection images), and evaluate the outcome of the intervention (resulting in a 3D volumetric image), where all imaging steps are performed on the same system. Furthermore, the system provides functionality for patient table motion, gantry motion, and the like, such that the interventional device is always in the field of view. Imaging parameters (e.g., x-ray technique, view angles, and the like.) can be either user-driven, protocol-based, or combinations thereof. These and other aspects of the present disclosure are described in more detail herein below.

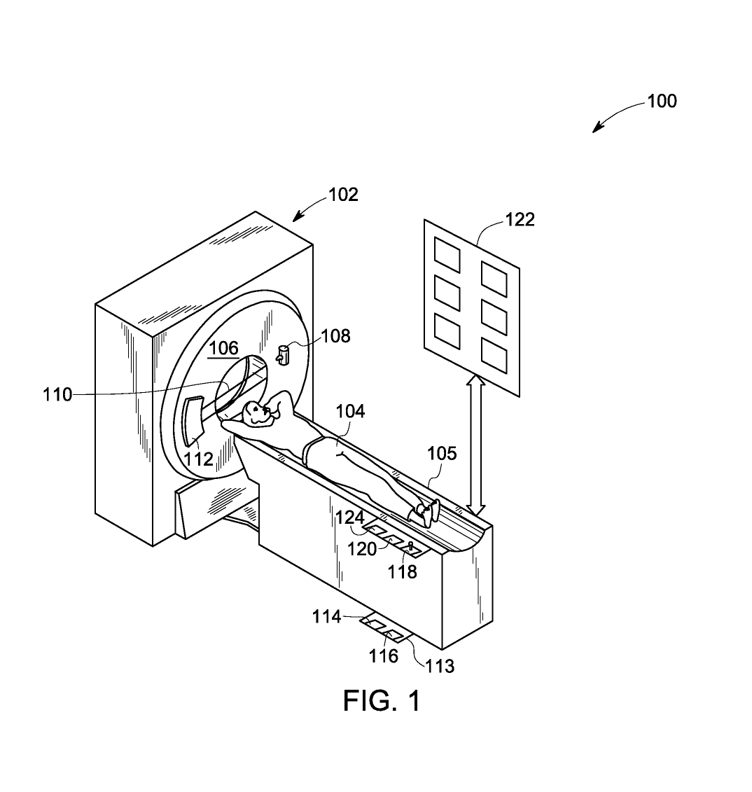

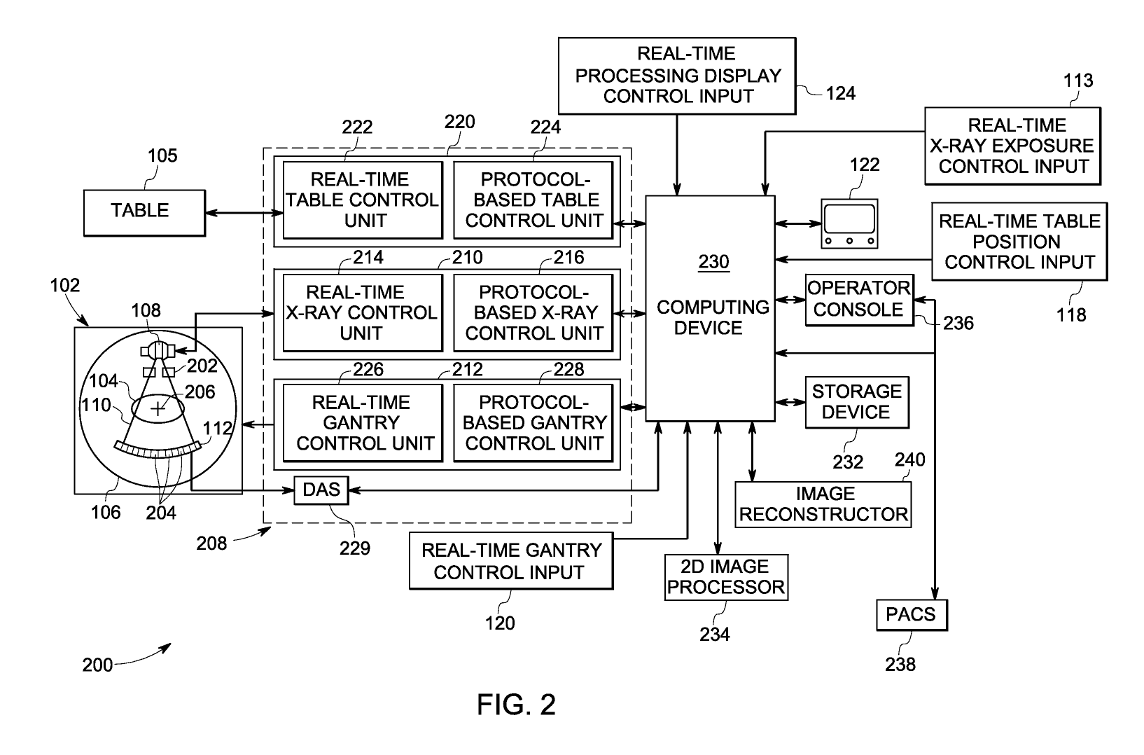

For discussion purposes, embodiments of the present system are described with reference to the integral CT/I system. However, in certain embodiments, the present system may include any other imaging device capable of sub-second scanning at a plurality of angles around a subject for whole organ imaging and incorporating a large-area detector for imaging the subject in different operating environments. As used herein, the term “large-area detector” refers to one or more detectors that, individually or in combination, provide adequate coverage for 2D and/or 3D imaging of the pathological area of interest within organs such as the heart, liver, brain, or vascular system of a patient. The detectors in combination may provide a square, rectangular, or other polygonal shapes, and may be flat or curved. Further, the detector may be read out at various resolutions (for example, using methods to combine signals from adjacent detector cells) as a function of the requirement of the 2D or 3D imaging being performed. Various implementations of the present systems and methods for allowing simultaneous diagnostic and interventional imaging will be described in greater detail with reference to FIGS. 1-2.

FIG. 1 illustrates an exemplary imaging system 100, for example, for use in interventional medical procedures. In one embodiment, the system 100 may include an integral CT/Interventional (CT/I) system 102 configured to acquire projection data from one or more view angles around a subject, such as a patient 104 positioned on an examination table 105 for further analysis and/or display. The CT/I system 102 may incorporate features of a multi-slice and/or a volumetric CT system for obtaining high-fidelity functional and structural information from the patient 104. Moreover, the CT/I system 102 may offer a wide array of axial coverage, high gantry rotational speed, and high spatial resolution. In one embodiment, for example, the CT/I system 102 may use a cone-beam geometry to allow imaging of a volume, such as an entire internal organ of the patient 104 at high or low gantry rotational speeds.

To that end, the CT/I system 102 may include a gantry 106 and at least one radiation source 108 such as an x-ray tube for projecting a beam of x-ray radiation 110 towards a detector 112, for example, positioned on the opposite side of the gantry 106. In certain embodiments, multiple radiation sources may be employed to project a plurality of x-ray beams 110 for acquiring image data from different angular positions around the patient 104. In certain other embodiments, the radiation source 108 may be a distributed source configured to emit x-ray beams 110 from multiple focal spot locations, where the multiple focal spot locations may form a surface. To that end, the distributed radiation source 108, for example, may include one or more addressable solid-state emitters arranged in one or multi-dimensional field emitter arrays configured to emit the x-ray beams 110 towards the detector 112 for imaging the patient 104 from multiple angular positions.

In certain embodiments, the projection data corresponding to the target ROI may be acquired from the multiple angular positions using a single large-area detector 112 in one scan cycle. To that end, in one embodiment, the large-area detector 112 may include sections of high-resolution, for example, including detector cells of about 100-200 micrometers in size and relatively low-resolution sections, for example, detector cells of about 1 millimeter in size. In another embodiment, multiple smaller area detectors may be operated simultaneously and measured signals may be combined by analog and/or digital means to form the larger area detector 112 for providing improved performance, such as reduced read-out time. In certain embodiments, the large area detector 112 includes a combination of one or more energy-integrating (EI) and energy-discriminating (ED) detector elements (not shown) arranged in one or more desired configurations for characterizing tissue types, for example, using dual-energy imaging principles, thereby aiding in patient diagnosis. In certain other embodiments, the detector elements in the detector 112 may differ in size and/or energy sensitivity such that one or more of the detector elements may be configured for scanning a desired FOV of the CT/I system 102 at a desired resolution based on imaging requirements.

In one embodiment, for example, the large-area detector 112 allows full FOV imaging for generating high-fidelity cross-sectional images that provide both anatomical and functional assessment of the patient 104. In another embodiment, the large-area detector 112 allows imaging of the ROI to generate cross-sectional images of an organ under examination by selectively using the high-resolution detector elements. These high-resolution detector elements provide acquisition of high-resolution 2D projection images to facilitate interventional aspects of the imaging procedure. Thus, unlike conventional interventional imaging systems that typically acquire only a limited imaging volume corresponding to the pathological area of interest, use of the large-area multi-resolution detector 112 allows acquisition of projection data, corresponding to larger imaging volumes that may cover the entire area of interest, at a desired resolution.

Specific scanning parameters, such as relating to the gantry 106, the radiation source 108, the detector 112 and other components of the CT/I system 102 for imaging the patient 104, however, may depend upon designated mandates, imaging protocol and/or user requirements. Accordingly, in certain embodiments, the CT/I system 102 may include circuitry such as table-side controls for controlling the scanning parameters, for example, in real-time via user-input and/or based on designated imaging protocols being used by the CT/I system 102 during diagnostic and/or interventional imaging. For example, in one embodiment, the table-side controls may include a mechanism for real-time x-ray exposure control input 113, where the mechanism includes foot pedals 114 and 116, which may be configured to control high-exposure (record-mode) and low-exposure (fluoro-mode) operation of the x-ray source 108, respectively, based on real-time user input. In a further embodiment, the table-side controls may include one or more mechanisms for receiving a real-time table position control input 118 and a real-time gantry control input 120 for user-controlled table motion and/or position and gantry motion and/or position, respectively. The CT/I system 102, thus, may be configured to allow adaptive and/or interactive control of imaging and processing parameters using the various input mechanisms. Such adaptive or interactive control of various components of the CT/I system 102 using the various input mechanisms will be discussed in greater detail with reference to FIG. 2.

Further, in one embodiment, the projection data acquired by the detector 112 may be processed for further evaluation and/or display. The CT/I system 102 may use the processed projection data for providing an analysis of functional and structural characteristics corresponding to the target ROI of the patient 104. Additionally, the CT/I system 102 may also detect a pathological region of the patient 104 and reconstruct corresponding 2D and/or 3D images for use during an interventional procedure. In certain embodiments, the CT/I system 102 may be configured to selectively display the 2D projection images, the 3D cross-sectional images and/or the functional information based on real-time and/or protocol-based inputs. To that end, in one embodiment, the CT/I system 102 may include a mechanism for receiving a real-time processing display control input 124 configured to control a selective display of the 2D images, 3D images, combined images, and/or functional analysis, for example, based on user inputs and/or a specific imaging protocol being used. Exemplary configurations and functions of the real-time processing display control input 124 will be described in greater detail with reference to FIG. 2.

In certain embodiments, the CT/I system 102 may be configured to continually generate, transmit, and/or display one or more 2D projection images in real-time on an associated display 122 based on the real-time processing display control input 124, for example, to aid in navigation of an interventional device during the interventional procedure. Similarly, the CT/I system 102 may also be configured to generate and display 3D cross-sectional images or analyses of the acquired data pertaining to the region of interest on the display 122 for facilitating assessment of anomalies and/or the efficacy of the interventional procedure. In one embodiment, for example, the CT/I system 102 may enable verification of functional information such as improved tissue perfusion, which may result from an angioplasty and/or stent placement procedure. In another embodiment, the CT/I system 102 may facilitate planning of the interventional procedure such as volumetric imaging of the aortic root to facilitate placement of an aortic valve during a TAVI procedure.

Accordingly, in certain embodiments, the CT/I system 102 allows for appropriate positioning of an interventional device such as a catheter adapted for use in a confined medical or surgical environment such as a body cavity, orifice, or a blood vessel. Generally, the catheter may serve as a medium for delivering a contrast agent and/or devices such as stent, balloon, coil, clip, and/or a rotoblade inside the patient's vasculature. The catheter may also serve to administer an x-ray contrast agent into or proximal the target ROI of the patient 104.

In certain embodiments, the CT/I system 102 may be configured to acquire projection data from a plurality of view angles around the patient 104 sufficient to reconstruct high-fidelity 3D images. In one embodiment, projection data from varying angular ranges may be used to reconstruct the 3D data, or to provide 3D information about the image ROI (e.g., via stereo-viewing). In other embodiments, the CT/I system 102 may be configured to generate a plurality of 2D projection images at a defined number of positions about the patient 104. Particularly, in one embodiment, the CT/I system 102 may use the 2D projection data to locate and navigate a catheter towards the target ROI. Additionally, the CT/I system 102 may be configured to generate multiple projection images acquired at a subset of the view angle positions at multiple electrocardiogram (ECG)-gated time points that allow for rapid, high-fidelity assessment of wall motion over the whole heart. Such real-time wall motion estimation of the whole heart using the CT/I system 102 is not achievable using existing interventional systems.

The CT/I system 102 may also be configured to acquire volumetric projection data for use in 3D cross-sectional imaging, and anatomical and functional assessment of an imaging volume. A volumetric projection data set allows high-fidelity reconstruction of 3D images, whereas 2D projection data may include a subset of the volumetric projection data set. The volumetric projection data may also be processed to provide volumetric images and/or derived data such as mean transit time, mean blood flow, and blood volume, all of which may be presented to the interventionalist on the display 122 before, during, and/or after the interventional procedure. In certain embodiments, the display 122 may include a plurality of sub-displays for selectively displaying reconstructed images, analysis, and/or patient information from prior examinations. In one embodiment, the 2D projection images may be further processed by the CT/I system 102 and/or combined with the 3D cross-sectional images. Particularly, the CT/I system 102 may be configured to display the combination of images over a reference grid in a common coordinate system in real-time to aid the interventionalist in more accurately tracking movement of the catheter.

In one embodiment, the CT/I system 102 may generate a 2D projection image approximately every 33 ms or a 3D cross-sectional image approximately every few seconds for real-time guidance during interventional procedures. The continual generation of the 2D and/or 3D images and corresponding data allows for continual refreshment of images representing the target ROI. This continual refreshment allows the interventionalist to monitor not only structural changes indicative of progress of an endovascular tool, but also functional parameters corresponding to the target ROI in real-time. The monitored information may then be used for preparing for a diagnostic procedure prior to the intervention or to evaluate progress during the intervention.

Particularly, using the 2D data in combination with the 3D information during the interventional procedure provides diagnostic information and high-fidelity high-contrast images to allow accurate catheter or device placement and assessment of the therapeutic efficacy of the intervention in real-time, respectively. Certain exemplary configurations of an imaging system that greatly benefit interventional procedures using the 2D and 3D data acquired by the CT/I system 102 in accordance with aspects of the present disclosure will be described in greater detail with reference to FIG. 2.

FIG. 2 is a diagrammatic illustration of exemplary components of a system 200 such as the system 100 of FIG. 1 for performing integral CT/I procedures. The radiation source 108 projects, for example, fan-shaped or cone-shaped x-ray beams 110 for imaging the desired FOV of the patient 104. Particularly, the CT/I system 102 configures one or more parameters of the radiation source 108 to focus the x-ray beams 110 on the target ROI based on a designated configuration of the detector 112 and/or a desired imaging protocol.

Accordingly, in one embodiment, the CT/I system 102 includes a collimator 202 positioned proximate the radiation source 108 to define the size and shape of the one or more x-ray beams 110 that pass through a desired region of the patient 104. The collimator 202 collimates the x-ray beams 110 based on specific imaging or application requirements using one or more collimating regions, for example, defined using lead or tungsten shutters. In certain embodiments, the collimator 202 collimates the x-ray beams 110 for reducing the x-ray dose administered to the patient 104 and reducing scatter radiation to the medical practitioner. The collimated x-ray beams 110 pass through the patient 104 and are attenuated by the patient's anatomy. The unattenuated portion of the x-ray beams 110 impacts one or more regions of the detector 112.

In one embodiment, the detector 112 may include a single large-area detector that provides coverage over the entire region of interest such as heart, liver, or brain in a single rotation of the gantry 106. Alternatively, the detector 112 may include a high-resolution section, such as a flat-panel detector adapted to provide high-resolution projection data over a large area. In a further implementation, the detector 112 may include a plurality of detector elements 204 that together sense the projected x-ray beams 110 that pass through the patient 104. Particularly, in one embodiment, two or more of the detector elements 204 may be operated simultaneously to measure signals that may be combined by analog or digital means to approximate the larger area detector elements (not shown).

As previously noted with reference to FIG. 1, in one embodiment, the detector 112 may correspond to a hybrid detector that includes a combination of one or more EI and ED detector elements 204 arranged in one or more desired configurations to provide adequate coverage for imaging a pathological area. The EI detector elements in the hybrid detector 112 produce an electronic signal proportional to the total amount of absorbed x-ray energy during each view. Further, the ED detectors may provide information regarding the energy distribution of the detected photons by producing two or more signals corresponding to two or more energy bins, for example, a high-energy bin and a low-energy bin.

The energy distribution information is useful for characterizing tissue type, which may be useful for diagnosis of disease affecting the patient 104. Furthermore, in certain embodiments, the detector elements 204 in the hybrid detector 112 may differ in size and/or energy sensitivity such that one or more of the detector elements 204 may be configured for scanning a desired FOV of the patient 104 at a desired resolution. To that end, in one example, a portion of the hybrid detector 112 having smaller detector elements may be selectively operated for generating high-resolution 2D projection images, whereas larger detector elements may be selectively operated for generating high-fidelity 3D volumetric images. Alternatively, the signals of smaller detector elements may be combined to form larger detector elements prior to signal digitization thereby resulting in a faster readout needed for 3D volumetric acquisitions. In certain embodiments, the detector 112 may include detector elements 204 that all have the same resolution.

Generally, the detector elements 204 provide electrical signals corresponding to the intensity of transmitted x-ray beams 110 at one or more angular positions around the patient 104 for collecting projection data for use in 2D image presentation or 3D image reconstruction. Accordingly, during a preliminary scan, the gantry 106 and the components mounted thereon may rotate about a center of rotation 206. In one embodiment, the system 200 may include a control mechanism 208 that may be configured to control the rotation of the gantry 106 and the operation of the radiation source 108 based on desired scanning requirements and/or examination protocols. The control mechanism 208, for example, may include an x-ray controller 210 that provides power and timing signals to the radiation source 108 and a gantry motor controller 212 that be configured to control the rotational speed, tilt, view angle, and/or position of the gantry 106 based on scanning requirements.

To that end, the x-ray controller 210 may include a real-time x-ray control unit 214 and a protocol-based x-ray control unit 216. The real-time x-ray control unit 214 manages x-ray exposure in real-time using the real-time x-ray exposure control input 113 during the medical procedure based on user input. For example, in one embodiment, the foot pedals 114 and 116 (see FIG. 1) may be employed to control high-exposure (record-mode) and low-exposure (fluoro-mode) operation of the x-ray tube. For these and other imaging modes, the real-time x-ray exposure control unit 214 may be configured to optimize imaging parameters, for example, voltage (kVp) and current (mA) configuration values, rate of mA and kVp switching are adjusted based on specific imaging scenarios.

In certain embodiments, the x-ray controller 210 may be configured to use the protocol-based x-ray control unit 216 for controlling the x-ray exposure based on a designated imaging protocol, such as acquisition of projection data for both 2D and 3D image generation. During diagnostic CT imaging, for example, the protocol-based x-ray exposure control unit 216 may be configured to maintain a designated kVp, while varying the mA over the scan duration to acquire projection data for generating images of a desired quality or at a desired radiation dose level. However, in an embodiment where the CT/I system 102 is operating in the 2D interventional imaging mode, the real-time x-ray exposure control unit 214 may be configured to vary both the mA and kVp to optimize image quality as the interventionalist pans the CT/I system 102 over the patient's body.

Alternatively, while imaging the patient 104, the interventionalist may adjust the mA, kVp and/or FOV using the real-time x-ray exposure control input 113 if patient and/or gantry motion is detected. Generally, when using iodinated contrast agents, higher attenuation is achieved with lower kVps. Accordingly, in one exemplary implementation, the real-time x-ray exposure control unit 214 may be configured to minimize kVp and correspondingly increase mA, for example, using a priori information. If a desired image quality is not achieved for a given tube voltage setting (kVp) when using a maximum beam current, the real-time x-ray exposure control unit 214 may be configured to increase kVp, thereby resulting in improved image quality at the cost of reduced contrast between the iodine and a tissue of interest. In certain embodiments, the real-time x-ray exposure control unit 214 may be configured to perform exposure management in real-time time during scanning, for example, at a rate of more than 30 times per second, depending upon a frame rate used for imaging during a particular procedure.

Additionally, the CT/I system 102 may also include a table motor controller 220, which allows control of the position and/or orientation of the table 105. To that end, the table motor controller 220 may include a real-time table control unit 222 and a protocol-based table control unit 224 configured to move the table 105 for appropriately positioning the patient 104 in the gantry 106. The real-time table control unit 222 may rapidly respond to the real-time table position control input 118 received from the interventionalist, for example, via the table-side controls, such as a joystick, to allow real-time changes in table orientation and positioning. Particularly, the real-time table control unit 222 may move the table 105 forward, backward, right, left, or may tilt the table 105 to position the patient 104 within the FOV of the CT/I system 102 in real-time based on operator supplied commands and parameters.

During interventional imaging, for example, the real-time table position control unit 222 may be configured to move the table 105 to right, left, forward, backward and or in a tilted position with respect to the reference position. The interventionalist may grossly position an interventional device in the patient 104 in the FOV of the CT/I system 102 by moving the table 105 using the real-time table position control unit 222. Once the interventional device can be visualized, the interventionalist advances position of the interventional device within the vasculature and performs a diagnostic procedure or a therapeutic procedure. The real-time table position control unit 222, thus, may be configured to allow for a greater range of patient positions for facilitating the intervention at a target region of interest (ROI) in all kinds of patients and/or modifying the FOV for acquiring projection data from a plurality of angular positions around the patient 104.

In certain embodiments, the table position and orientation may be progressively modified based on the specific imaging and/or examination protocol being performed. Specifically, in examinations that entail imaging large anatomical regions, the protocol-based table control unit 224 may adjust position of the table 105 at various stages of the examination to position a specific region of the patient 104 within the desired FOV of the CT/I system 102. For example, in CT imaging mode, when generating 3D images of the region of interest, the protocol-based table position control unit 224 may be configured to move the patient table 105 into the gantry 106 or out of the gantry 106, and/or position the gantry 106 in a tilted position with respect to a reference position. Particularly, the table position may be modified to facilitate acquisition of the necessary projection data such as is the case for axial step-and-shoot and helical data acquisition protocols.

Moreover, in certain embodiments, the rotation, orientation, and position of the gantry 106, and in turn, the x-ray source 108 and the detector 112 may be controlled using the gantry motor controller 212. In one embodiment, the gantry 106 may be moved forward and backward to position the CT/I system 102 with respect to the patient 104 on the table 105 via rails or other mechanical mechanisms. Further gantry view angle and tilt may be used to specify the view angle of projection images. To that end, the gantry motor controller 212 may further include a real-time gantry control unit 226 and a protocol-based gantry control unit 228 that allow acquisition of projection data based on real-time input and protocol-based specifications, respectively. In certain embodiments, the real-time gantry control unit 226 may be configured for selectively positioning the gantry 106 based on imaging requirements. In one embodiment, the real-time gantry control unit 226 may use a gantry control input 120 for receiving specification of gantry view angle, tilt, and position for real-time control of the gantry orientation relative to the patient 104. Use of the real-time gantry control input 120, thus, allows imaging of the patient 104 from desired angular positions, for providing the interventionalist with the desired projection of the target ROI. Further, the real-time x-ray control unit 214 may also be configured to control the collimator 202 for defining the size and shape of the x-ray beams 110 passing through a desired region of the patient 104 based on imaging specifications. By way of example, the imaging specifications may include a mandate for reducing radiation dose to the patient 104 and/or the interventionalist during data acquisition by the detector elements 204.

Further, the protocol-based gantry control unit 228 may be used to control operation of gantry 106, for example, during acquisition of 3D cross-sectional imaging data. Specifically, the protocol-based gantry control unit 228 may be configured to control a rotation speed of the gantry 106 and the duration of the gantry rotation, while the CT/I system 102 acquires projection data for 3D imaging purposes.

Furthermore, in certain embodiments, the control mechanism 208 may also include a data acquisition system (DAS) 229 for sampling analog data from the detector elements 204 and converting the analog data to digital signals for subsequent processing. Moreover, in certain embodiments, the data sampled and digitized by the DAS 229 may be input to a computing device 230. The computing device 230 and/or a 2D image processor 234 may be configured to process and display the 2D projection data with sufficiently low latency to enable eye-hand coordination for interventional device guidance. In another embodiment, 3D volumetric images, or volumetric images of small ROIs (e.g., centered around the tip of the catheter or centered around the device), and/or 3D volumetric images based on reconstructions from small angular ranges (e.g., less than 180 degrees) are provided in real-time, or near real-time to the user. Additionally the computing device 230 may store this data in a storage device 232, such as a hard disk drive, a floppy disk drive, a compact disk-read/write (CD-R/W) drive, a Digital Versatile Disc (DVD) drive, a flash drive, or a solid-state storage device. In certain embodiments, the computing device 230 may include modules and/or applications that allow for automated analysis of the acquired image data, for example, for estimating location of a target ROI for further evaluation and/or extraction of anatomical and/or functional information associated with the target ROI.

In one embodiment, the target ROI may correspond to at least a portion of a desired area of interest of the patient 104 that was evaluated during a preliminary scan. The preliminary scan may provide diagnostic images and related information for identifying the target ROI that is indicative of diseased tissues. Typically, at least a portion of biological tissues may exhibit different or abnormal characteristics in comparison to normal tissue. The preliminary scan may allow identification of such diseased tissues, which exhibit abnormal composition, density, shape, size, structure, and/or function indicating presence of a diseased state or medical condition such as the presence of atherosclerosis, cancer, tumor, fibrosis or stenosis. Accordingly, in one embodiment, the computing device 230 may analyze the preliminary scan data and/or images, for example, using automated analysis tools as these parameters are closely linked to tissue state with respect to pathology. In certain embodiments, the computing device 230 may use operator input in place of, or in addition to the preliminary scan data for identifying the target ROI.

To that end, in certain embodiments, the computing device 230 may be coupled to the display 122, such as one or more monitors, printers or display means integrated into wearable eyeglasses. The display 122, for example, may be table-mounted, ceiling-mounted or cart-mounted to allow an operator to observe real-time 2D projection images, reconstructed 3D images, or a combination of 2D and 3D images, as well as data derived therefrom, and other relevant information such as exposure time and/or contrast dose used at different points of time during the procedure. Particularly, in one embodiment, the display 122 may include an interactive user interface that may be configured to allow selection and display of scanning modes, FOV, prior exam data, and intervention path. The interactive user interface may also allow on-the-fly access to 2D and 3D scan parameters and selection of an ROI for subsequent imaging, for example, based on operator and/or system commands.

Accordingly, in one embodiment, the processing display control input 124 may be used to configure a selective display of the 2D, 3D and/or combined images and/or analysis on the display 122, for example, based on the specific imaging protocol being used. Use of the processing display control input 124 may allow control of the display 122 in real-time during the medical procedure to allow processing and/or display of desired images and/or information. In one example, the processing display control input 124 may be used to control the display of processed projection data for providing useful real-time visualization, digital subtraction angiography and geometric and/or functional analysis. In another example, the real-time processing display control input 124 may be used to control display of post-processed reconstructed images to focus on specific regions for assessing a nature and extent of disease in the patient 104. In a further embodiment, the real-time processing display control input 124 may allow fetching and display of patient data from associated sensors such as an ECG monitor, a magnetic resonance system, an ultrasound system (such as intravascular ultrasound), an optical imaging system (such as optical coherent tomography), or prior examination sequences stored on the 2D image processor 234 and/or a picture archiving and communications system (PACS) 238.

Accordingly, in one embodiment, the real-time processing display control input 124 may include devices such as a graphical user interface, a touch screen, a joystick and/or a table-side mouse. Additionally, the real-time processing display control input 124 may include menu and control options to allow the interventionalist, for example, to select and configure the x-ray imaging protocol, manage the radiation dose in real-time and/or indicate the FOV, gantry angular orientation, gantry tilt, gantry position, and other parameters for imaging during subsequent scans. Moreover, the real-time processing display control input 124 may also allow identification of a pathological ROI, display of the position of a catheter and/or surrounding tissues in relation to the ROI on the display 122, or navigation of the catheter past a tortuous section of vasculature.

Collectively, the real-time radiation exposure control input 113, the real-time table position control input 118, the real-time gantry control input 120, and the real-time processing display control input 124 allow for real-time specification of x-ray technique (for example, current, voltage, and pulse width setting of the -ray source 108), table motion/position, gantry motion/position, and display of real-time processing, post-processing, playback, and retrieving/viewing of 2D and 3D image data, respectively. In one embodiment, the real-time inputs may be provided to the CT/I system 102, for example, using inputs 113, 118, 120 and 124 received via the table-side controls depicted in FIG. 1.

For protocol-based procedures such as 3D imaging, in certain embodiments, the operator may specify commands and scanning parameters via an operator console 236, which may include a keyboard (not shown). To that end, the operator console 236, for example, may include a panel that includes mechanisms for receiving the real-time inputs 113, 118, 120 and 124 depicted in FIG. 1. The panel may use the real-time inputs 113, 118, 120 and 124 for configuring the control mechanism 208 to control fluoroscopy exposure, CT exposure, table motion and orientation, gantry motion and orientation, and transmitted radiation.

Although FIG. 2 illustrates only one operator console 236, more than one operator console may be coupled to the CT/I system 102, for example, for inputting or outputting system parameters, requesting examinations and/or viewing images. Further, in certain embodiments, the system 200 may be coupled to multiple displays, printers, workstations, and/or similar devices located either locally or remotely, for example, within an institution or hospital, or in an entirely different location via communication links in one or more configurable wired and/or wireless networks such as a hospital network and virtual private networks. In one embodiment, for example, the computing device 230 may be coupled to PACS 238. In an exemplary implementation, the PACS 238 is further coupled to a remote system such as a radiology department information system, hospital information system and/or to an internal or external network (not shown) to allow access to the image data.

Further, the CT/I system 102 may be used to provide imaging data when an interventional device, for example a catheter, is inserted into the patient's body. To that end, in one example, the interventional device may be inserted into the patient's body via an access site different from the area of interest to allow the interventionalist to perform the interventional procedure at the target ROI without obstructing the FOV of the CT/I system 102 configured to image the target ROI. To that end, the right radial or brachial artery in the arm or the femoral artery in the groin region of the patient 104 may be punctured using the interventional device such as a needle. Additionally, a catheter may be inserted into the puncture site using a guide wire (not shown) and advanced towards the target ROI, for example, in the cardiac, hepatic or cranial region.

The navigation of the catheter within tortuous portions of the vascular system can be difficult using conventional 2D fluoroscopic systems. In order to provide both interventional and CT imaging in a single setting, currently available systems merely position an interventional system and a CT system in a single room shuttling the patient table between the two imaging systems. Such conventional systems, however, occupy a large amount of floor space and incur significant time delay and patient/table motion to switch imaging systems. Accordingly, switching between interventional and CT imaging only occur at “safe” or “rest” points in the procedure, and may be of limited utility for interventional procedures requiring the integration of the two imaging modalities. Further, the time delay incurred in transporting the patient 104 between the diagnostic and interventional systems increases scanning time, time under anesthesia and/or retention period of interventional devices in the vasculature, increasing patient risk. Additionally, increased procedure time results in fewer procedures, which in turn, leads to loss of revenue.

In contrast to such conventional systems that physically combine two physically separate and distinct imaging modalities, embodiments of the system 100 employ the single CT/I system 102 to provide integrated CT and interventional imaging using the large area detector 112. To that end, in one example, a contrast agent, for example including an x-ray opaque iodine-based colorless “dye,” is injected into the vasculature using a catheter to accentuate the absorption of x-rays, thus improving visualization of blood vessels in the vicinity of the catheter tip. Further, the CT/I system 102 may be configured to continually measure 2D projection data and present one or more contrast-enhanced images of the patient 104 on the display 122 in real-time, and/or to evaluate 2D/3D information for presentation on the display 122 based on clinical requirements.

To that end, in one embodiment, the CT/I system 102 includes a 2D image processor 234 for reconstructing high-fidelity 2D images in real-time for use during the interventional procedure. By way of example, the 2D image processor 234 may process the projection data to analyze the 2D images for tracking movement of the interventional device within the patient's body in real-time. Generally, for hand-eye coordination, it is desirable that the time starting from the instant at which the interventional device is moved, the detector 112 is read out, the acquired data is processed and subsequently loaded onto the display 122 for display is less than 150 ms. Use of the dedicated 2D image processor 234 allows for a separate 2D image processing chain that aids in generating low-latency 2D images and corresponding diagnostic information. The low-latency 2D images and/or information, in turn, may be advantageously used for real-time guidance of the interventional device, thus providing functionality typically not available with conventional systems.

Further, in one embodiment, the 3D cross-sectional images may be generated as per an imaging requirement by an image reconstructor 240 operatively coupled to the computing device 230. By way of example, during a cardiac evaluation, the image reconstructor 240 may be configured to generate one or more 3D images of the heart for display on the display device 122 for evaluation and/or intervention. Particularly, in one embodiment, the generated images may be used for diagnosing whether functional ischemia is associated with a cardiac stenosis by estimating heart wall motion and/or tissue perfusion parameters.

One of the goals of all non-invasive imaging for functional assessment is to identify the patients that will benefit from the percutaneous coronary intervention in the catheterization laboratory. Generally, the anatomic assessment and limited functional assessment is performed using a CT or an MR imaging system. The CT/I system 102, however, allows the anatomic and functional assessment, and catheterization to be performed using a single system. Particularly, the CT/I system 102 allows for complete functional assessment of the myocardium by estimating wall motion using the plurality of 2D projection data acquired at multiple ECG-gated time points and multiple angular positions. Further, as previously noted, the CT/I system 102 allows heart-wall motion measurements, for example, via single- or multiple-energy ECG-gated projections at a plurality of determined angles using the large-area detector 112. During one or more cardiac cycles, multiple 2D projection data may be acquired at a plurality of view positions about the heart. The sequence of 2D projection data at each of a plurality of view positions is used to estimate wall motion over the whole volume of the heart, such as in the ventricles and atria. A benefit of this procedure is that a CT contrast agent, such as an iodine-based agent, may be injected in a peripheral vein in the arm, whereas during an interventional procedure, arterial injections of a contrast agent via an inserted catheter is used to improve vessel opacification. The interventional procedure is highly invasive, adding risk to the medical procedure.

Although the CT/I system 102 is described with respect to cardiac imaging, it may be noted that the CT/I system 102 may also be configured to acquire one or more projection data at one or more view angles during a sufficient temporal acquisition window to allow functional analysis within the ROI, where the ROI may include any location in the body.

Accordingly, in one embodiment, the CT/I system 102 may determine the angles as well as strategic time points during the course of the heart cycle within a specific ROI. Specifically, the CT/I system 102 may acquire projection data at the determined time points and angular positions for generating contrast enhanced images. Further, the CT/I system 102 may use the contrast enhanced projection images for estimating the heart wall motion. The estimated heart wall motion, in turn, may be used by the CT/I system 102 to determine possible local cardiac ischemia caused by a stenosis and to assess if revascularization may provide clinical benefit. Due to the fast rotation speed of the gantry 106 and the whole-organ coverage provided by the large area detector 112, the plurality of projection data acquired at the plurality of angular projections may be acquired during a single cardiac cycle or multiple cardiac cycles.

Typically, the ability to assess functional ischemia associated with a stenosis allows for a more informed determination regarding which patients should undergo catheterization. Conventionally, functional assessment is performed by measurement of fractional flow reserve during the interventional procedure (invasive techniques) or with nuclear imaging such as using positron emission tomography (PET) or single photon emission computed tomography (SPECT). To that end, the interventionalist positions a catheter that includes a pressure sensor both distal and proximal to a lesion while recording pressure measurements to estimate fractional flow reserve. The ratio of the distal to proximal pressure measurements provides a discriminator for identifying lesions requiring therapy, such as stent placement.

However, in certain embodiments, the interventionalist may use the CT/I system 102 to identify and revascularize one or more ischemia-causing stenoses using the real-time 2D projection images without moving the patient 104. The interventionalist determines if the catheter tip is proximate the target ROI based on the 2D projection images. Alternatively, the computing device 230 may be employed to analyze the projection images to determine the location of the catheter tip in the patient's body. In one embodiment, if the catheter tip is determined to be located at a distance that is greater than a desired distance from the target ROI, the computing device 230 may configure the gantry motor controller 212 and/or the table motor controller 220 to align the patient 104 suitably for imaging. Alternatively, the CT/I system 102 may allow the interventionalist to adjust an imaging axis manually. Particularly, the patient 104 is aligned such that at least the catheter tip is visible in the displayed 2D projection images.

Once the catheter tip is visible in the projection images, the CT/I system 102 may be configured to acquire data for 2D images and/or 3D cross-sectional images according to the examination and/or interventional procedure being undertaken. In one embodiment, the CT/I system 102 generates 2D images for catheter guidance, whereas the 3D cross-sectional images may also be used for device guidance or to provide diagnostic information and/or relating functional assessment and may be generated at any point during the interventional procedure. The CT/I system 102, for example, acquires data for generating the 3D cross-sectional images used in sizing and placing a valve during TAVI or for verifying an improvement in perfusion once the interventional procedure is complete.

Furthermore, in certain embodiments, a combination of the 2D and/or the 3D images may be continually updated on the display 122 within the ROI. To that end, the 2D and 3D images may be registered in a common coordinate system using a transformation that aligns the 2D and 3D images based on one or more designated fiducials, such as anatomical landmarks, the table 105, gantry position or the catheter positioned within the patient's body. The registration of the 2D and 3D images may be performed automatically or based on user input via the table-side controls. The combined 2D and 3D images allow the interventionalist to verify in real-time a current location of a catheter in relation to the target ROI and/or the surrounding tissues while guiding the catheter along the vasculature towards the target ROI. In such a scenario, the 3D cross-sectional images may provide help for navigating tortuous vessels.

It may be noted that although embodiments of the present system 200 are described with reference to an integral CT/I system, in certain embodiments, the system 200 may facilitate other medical procedures and incorporate additional imaging modalities. These auxiliary imaging modalities include, for example, imaging with an optical coherence tomographic system, an ECG monitor, or an intravascular ultrasound system.

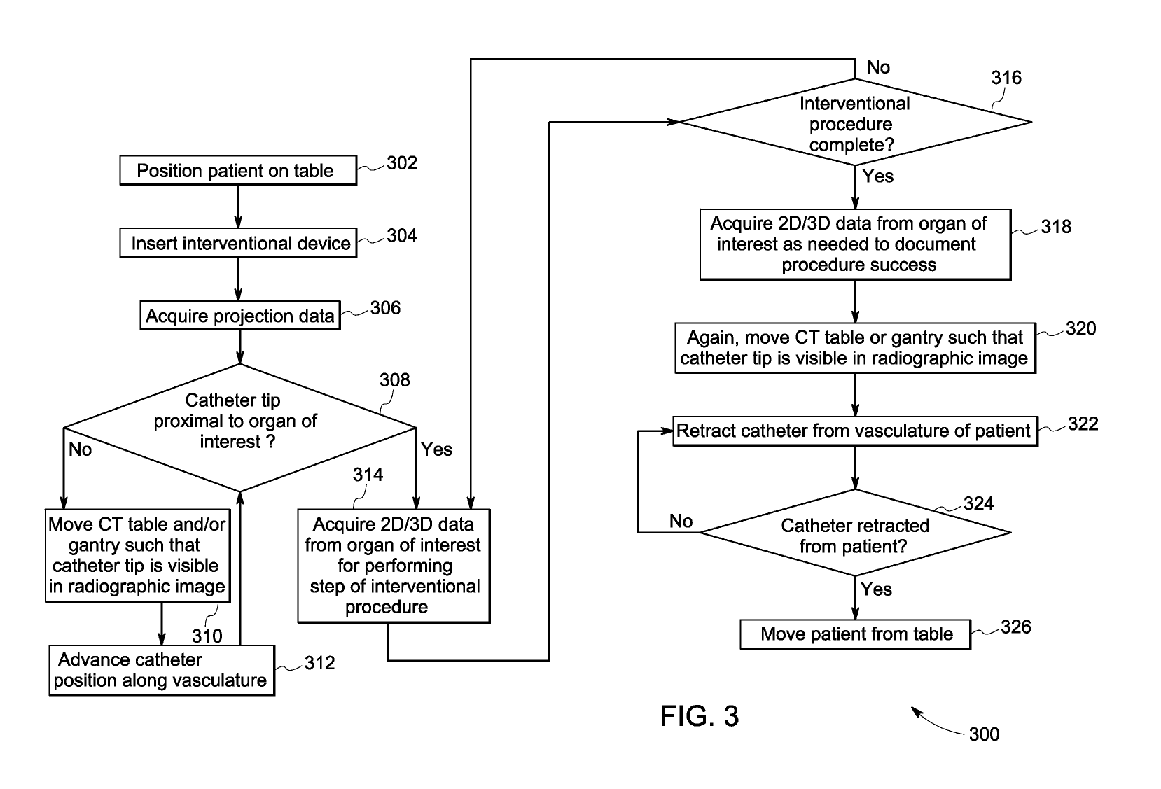

Generation of high-fidelity 2D and 3D images and related information as per a configuration input in real-time during a medical procedure provides a great amount of accuracy, flexibility and adaptability for accommodating different medical procedures on the same CT/I system 102 without repositioning or transporting the patient 104. Particularly, use of embodiments of the system 100 allows for integrated high-fidelity 3D cross-sectional imaging and 2D projection imaging for facilitating interventional procedures, thus reducing the overall system complexity, physical footprint, and other specific imaging parameters such as examination time, radiation, and/or contrast dose administered to the patient 104. The reduction in values of specific imaging parameters, in turn, provides added system flexibility to the interventionalist, while improving patient care, safety, and comfort. Additionally, the ability to perform simultaneous imaging and intervention obviates use of a separate interventional suite, further curtailing equipment, and examination costs. Certain exemplary methods for performing interventional procedures using the integral CT/I system, such as the CT/I system 102 of FIGS. 1-2, will be described in greater detail with reference to FIG. 3.

FIG. 3 illustrates a flow chart 300 depicting an exemplary method for imaging a target ROI of a patient for facilitating both diagnostic and interventional procedures using the same imaging system. Embodiments of the exemplary method may include computer executable instructions on a computing system or a processor. Generally, computer executable instructions may include routines, programs, objects, components, data structures, procedures, modules, functions, and the like that perform particular functions or implement particular abstract data types. Embodiments of the exemplary method including computer executable instructions may also be practised in a distributed computing environment where optimization functions are performed by remote processing devices that are linked through a wired and/or wireless communication network. In the distributed computing environment, the computer executable instructions may be located in both local and remote computer storage media, including memory storage devices.

Further, in FIG. 3, the exemplary method is illustrated as a collection of blocks in a logical flow chart, which include certain operations that may be implemented in hardware, software, or combinations thereof. The various operations are depicted in the blocks to illustrate the functions that are performed, for example, during image data acquisition, processing, image reconstruction and interventional phases of the exemplary method. In the context of software, the blocks represent computer instructions that, when executed by one or more processing subsystems, perform the recited operations.

The order in which the exemplary method is described is not intended to be construed as a limitation, and any number of the described blocks may be combined in any order to implement the exemplary method disclosed herein, or an equivalent alternative method. Additionally, certain blocks may be deleted from the exemplary method or augmented by additional blocks with added functionality without departing from the spirit and scope of the subject matter described herein. For discussion purposes, the exemplary method will be described with reference to the elements of FIGS. 1-2.

Generally, interventional procedures such as angioplasty are conventionally performed using 2D projection images for various diagnostic and/or therapeutic purposes. However, as previously noted, the 2D projection images may provide only limited information regarding the actual 3D anatomy, thus constraining a medical practitioner's understanding of a relationship between operations performed during the interventional procedure and the vascular anatomy. Certain clinical applications, however, use high spatial resolution images for investigating minute features within a patient, such as coronary vessels of a human heart. Particularly, accurate characterization of specific features, for example, corresponding to the thoracic cavity allows for a better understanding of the physiology of the heart.

To that end, certain conventional interventional systems may employ imaging systems such as C-arm systems to allow generation of pseudo-3D or 3D images. Conventional C-arm imaging generally entails multiple acquisitions from two or more view angles while the gantry is slowly rotating, typically at a rate of 180 degree plus fan angle rotation (−210 degrees) per 5 seconds, around the ROI. The long acquisition time may result in patient movement including breathing and cardiac motion, as well as changes in iodine contrast concentration that may lead to artifacts in reconstructed images. The suboptimal image quality, in turn, may impair determination of an appropriate diagnosis and treatment, thus endangering patient health.

Accordingly, embodiments of the present method describe techniques for enhanced imaging and interventional procedures using high-fidelity and low-latency 2D projection images in real-time and/or 3D cross-sectional images as needed during the procedure. To that end, at 302, a patient such as the patient 104 may be suitably positioned on an examination table such as the table 105 associated with the CT/I system 102 for imaging and/or performing diagnostic and therapeutic interventional procedures to a desired region, such as the patient's heart. Particularly, the patient is positioned such that the desired region may be positioned within an FOV of the CT/I system 102.

Further, at step 304, an interventional device, such as a catheter, may be inserted into a patient's vasculature via an access site distal from a target ROI where the target ROI encompasses an organ of interest of the patient. Alternatively, a needle may be inserted into a patient's body proximate to the target ROI. As previously noted, the target ROI and/or the organ of interest may be determined based on a preliminary scan or prior exam data. Generally, the interventional device may be inserted into a vein or artery in the patient's extremity distal from the target ROI to allow the interventionalist to perform the interventional procedure at the target ROI without obstructing the FOV of the CT/I system 102 configured to image the target ROI. Accordingly, in one embodiment, an interventional device such as a catheter is inserted into an artery or vein in the arm or in the groin region of the patient. Particularly, the catheter may be inserted into the insertion site using a guide wire and then be advanced towards the target ROI, for example, in the cardiac, hepatic, or cranial region. Although not specifically mentioned, any interventional device that facilitates an interventional procedure is within the scope of the present disclosure.

Further, at step 306, the CT/I system, using sub-second scanning, may acquire projection data for facilitating placement of the catheter within the patient based on specific imaging protocols. In certain embodiments, the catheter itself may facilitate its localization. Alternatively, a contrast agent may be administered into the vasculature of the patient, for example, via the catheter to improve visualization of the surrounding regions. The contrast agent increases opacification of the blood proximal to the administration site, and thereby facilitates the identification of the vasculature from nearby overlapping and confounding anatomical structures. Additionally, the contrast agent may be a fluid administered to the patient as an intravenous infusion at a steady state to provide a generally steady state of contrast, for example, for cardiac CT imaging, as described hereinabove. Alternatively, for applications such as perfusion studies, a bolus injection of the contrast agent may be administered, where a large quantity of the contrast agent is rapidly injected into the patient 104 via an intravenous site. For interventional procedures, contrast agents are generally delivered intra-arterially via the catheter.

In certain embodiments, the CT/I system 102 may acquire the projection data from the target ROI, for example, after the contrast agent has been administered. However, in other embodiments, the CT/I system 102 may acquire projection data suitable for 2D projection imaging and/or 3D cross-sectional imaging prior to or during contrast agent administration based on the specific imaging requirements of the procedure being undertaken. Particularly, the CT/I system 102 may acquire projection data suitable for use in generating high-fidelity 2D projection images, 3D volumetric images and/or for displaying corresponding structural and functional information in real-time. By way of example only, and not intended to be a limitation, in one embodiment, a real-time time frame corresponds to generation of a 2D projection image for catheter placement approximately every 33 ms with a time delay of approximately 150 ms. Further, projection data for 3D cross-sectional imaging in real-time, for example, may be acquired approximately every 350 ms, while reconstructing and displaying the volume in several seconds.

In certain embodiments, the CT/I system 102 may allow ‘on the fly’ customization of the imaging protocol before, during and/or after the procedure to switch between acquiring low-latency 2D projection data and 3D volumetric data. During cardiac imaging, for example, the CT/I system 102 may be configured to allow for ECG-gated triggering of data acquisition during a cardiac cycle. In certain embodiments, the CT/I system 102 may acquire projection data in a continuous acquisition mode to allow generation of a collection of 3D volumetric images that are representative of the dynamic state of an object, such as that exhibited by cardiac motion. For real-time catheter navigation, however, the CT/I system 102 may operate in a 2D data acquisition mode to generate 2D projection images.

Further, at step 308, one or more of the acquired projection data and/or corresponding images may be used to determine if the tip of the interventional device is proximal to the organ of interest. In one embodiment, the interventionalist may identify the location of the interventional device in the vasculature, for example, based on the 2D images rendered on the display, or by injection of a bolus of contrast agent to facilitate location of the catheter tip in the processed images. Alternatively, the computing device, such as the computing device 230 may identify the interventional device location based on automated analysis of the 2D images and/or the acquired projection data.

If it is determined that the tip of the interventional device is not proximal the organ of interest, at step 310, the computing device may reconfigure one or more parameters of the CT/I system 102 so as to allow visualization of the tip of interventional device in subsequent 2D images. Specifically, in one embodiment, the computing device may configure the control mechanism of the CT/I system 102 to automatically move an associated examination table and/or the gantry to modify the FOV of the CT/I system 102 such that the resulting images allow visualization of the tip of the interventional device in relation to the vasculature. In an alternative embodiment, the interventionalist may control the movement of the associated examination table, gantry, and/or the FOV of the CT/I system 102 manually. Once the tip of the interventional device is visible, at step 312, the interventionalist may advance the interventional device along the vasculature towards the target ROI under guidance of the 2D images. In another embodiment, the gantry 106 and/or the table 105 may be moved such that the region of interest (e.g., the tip of the interventional device) is imaged with the high-resolution component of the detector.

However, at step 308, if it is determined that the interventional device is positioned proximal to the organ of interest, at step 314, the computing device may configure one or more parameters of the CT/I system 102 to acquire projection data for 2D projection imaging or 3D cross-sectional imaging using the large area detector. The type of data acquired is based on desired examination and/or user requirements for diagnostic imaging and/or intervention at the target ROI. In one embodiment, for example, a plurality of projection data may be acquired at a plurality of angular positions during one or more cardiac cycles to estimate chamber wall motion at the plurality of angular positions. Such wall motion estimation capability may be achieved with or without a peripheral contrast injection or an intra-arterial contrast injection via the interventional device. It may be noted that such wall motion estimation using the projection data acquired within a single cardiac cycle is currently not achievable with any state-of-the-art interventional device, and provides an example of the unique characteristics of the integral CT/I system 102.

In certain embodiments, the acquired 2D and/or 3D data may be further processed to provide derived information useful for exam prescription, planning and monitoring the interventional procedure. In one example, the 2D data may be used for determining functional parameters such as blood flow for ascertaining efficacy of the interventional procedure. In another example, the 2D data may be used to facilitate placement of a balloon in a diseased vessel for opening an occlusion, such as in an angioplasty procedure for cardiac and neural applications. Additionally, the 2D data may also facilitate placement of a stent at a location of stenosis in a coronary vessel or carotid vessel, and/or placement of a clip in a location of an aneurysm, such as in the brain. The 2D data may further be used for local administration of thrombolytics to remove a clot such as in the brain, physical removal of a thrombosis at the site of occlusion and/or local administration of material to occlude vessels such as for tumor embolysis.

Similarly, the 3D cross-sectional images may be used in a plurality of medical scenarios. In one example, the 3D cross-sectional images may be employed to identify the location of coronary vessel ostia prior to an aortic valve replacement in a trans-aortic valve intervention. The 3D cross-sectional images may also be used for the identification of the 3D representation of vasculature such as during cardiac CT imaging and/or neural imaging. In certain scenarios, the 3D representation of the vasculature may be generated prior to insertion of the interventional device and with CT angiographic procedures, which utilize a peripheral injection of contrast agent, typically in the arm. One or more 3D images may be used for functional assessment of the anatomy, as with neuro and cardiac CT perfusion. It may be noted that the examples listed above for utilization of 2D and 3D imaging data are not meant to be limiting, but are intended to only provide examples of the broad-based applicability of the integral CT/I system 102 to different imaging scenarios.

Further, at step 316, the 2D and/or 3D images may be used to determine if the interventional procedure is complete. If the interventional procedure is not complete, control may be passed on to step 314 where the CT/I system 102 may continue imaging to allow control and tracking of the interventional device inside the vasculature for performing the interventional procedure. If it is determined that the interventional procedure is complete, at step 318, the computing device may configure the CT/I system 102 to acquire projection data from the target ROI to determine success or failure of the interventional procedure. For example, one or more 3D images of the target ROI may be generated to assess a change in functional and/or structural characteristics, such as, improvement in the perfusion values of the target ROI after the intervention.

If the assessed change is desirable, at step 320, the imaging parameters such as the position and/or orientation of the table and the gantry may be adjusted to visualize the tip of the interventional device in the projection images as discussed with reference to step 310. At step 322, the acquired 2D and/or 3D images may be used as guidance for retracting the interventional device from the patient's body. At step 324, a check may be carried out to determine if the catheter is retracted from the patient. If the catheter is not completely retracted from the patient, the interventionalist continues retracting the catheter under guidance of the 2D projection or 3D reconstructed images. Once the catheter is removed, at step 326, the patient may be moved from the table. As discussed previously, the incremental movement of the catheter and/or patient table may be accomplished by either computer control or manual control provided by the interventionalist.

It may be noted that although by way of example, use of a catheter is specifically mentioned in blocks 308, 310, 312, 320, 322, and 324 of FIG. 3, this example is not meant to be limiting. Use of any suitable interventional device is also envisioned within the scope of the present disclosure. Additionally, by way of example, a volume that includes an organ of interest is mentioned in blocks 308, 314, and 318 of FIG. 3. However, embodiments that include any volume that includes an ROI within the patient 104 are envisioned.

Embodiments of the present systems and methods, thus, aid in executing diagnostic imaging and interventional procedures with greater accuracy using high-fidelity 2D projection images and 3D cross-sectional images. Particularly, the embodiments described herein allow the interventionalist to use the images generated in real-time to assess and treat the patient during the interventional procedure with greater certainty. The 3D images not only allow accurate catheter positioning, but also aid in characterizing minute structural and functional characteristics of the pathology within the ROI with greater accuracy without having to move the patient between different systems. The accurate characterization, in turn allows the interventionalist to provide therapy to the exact location of the pathology and verify the efficacy of the therapy in real-time without risking patient health.

Particularly, as previously noted, use of the large-area detector in the CT/I system during these medical procedures allows imaging of the entire pathological area of interest in one scan cycle, thereby minimizing the imaging time, contrast medium dosage, radiation dosage, and the examination and equipment costs. Additionally, unlike conventional interventional systems, the embodiments of the present systems and methods allow for simultaneous imaging and intervention using the same system without entailing frequent patient repositioning, thus reducing the overall examination time and use of floor space, enhancing the productivity of the interventionalist and improving patient comfort.

It may be noted that the foregoing examples, demonstrations, and process steps that may be performed by certain components of the present systems, for example, by the control mechanism 208, the DAS 229 and computing device 230 may be implemented by suitable code on a processor-based system, such as a general-purpose or special-purpose computer. It may also be noted that different implementations of the present technique may perform some or all of the steps described herein in different orders or substantially concurrently, that is, in parallel. Additionally, the functions may be implemented in a variety of programming languages, including but not limited to Ruby, Hypertext Preprocessor (PHP), Perl, Delphi, Python, C, C++, or Java. Such code may be stored or adapted for storage on one or more tangible, machine-readable media, such as on data repository chips, local or remote hard disks, optical disks (that is, CDs or DVDs), solid-state drives, or other media, which may be accessed by the processor-based system to execute the stored code.

Although specific features of various embodiments of the present disclosure may be shown in and/or described with respect to some drawings and not in others, this is for convenience only. It is to be understood that the described features, structures, and/or characteristics may be combined and/or used interchangeably in any suitable manner in the various embodiments, for example, to construct additional assemblies and techniques.

While only certain features of the present disclosure have been illustrated and described herein, many modifications and changes will occur to those skilled in the art. It is, therefore, to be understood that the appended claims are intended to cover all such modifications and changes as fall within the true spirit of the present disclosure.

1. An imaging system, comprising:
an integral computed tomography and interventional system comprising a large-area detector configured to acquire projection data corresponding to a field of view of the imaging system from one or more view angles;
a computing device operatively coupled to the integral computed tomography and interventional system, wherein the computing device is configured to perform one or more of:
processing the projection data acquired by the large-area detector to generate a two-dimensional projection image in real-time;
processing the projection data acquired by the large-area detector to generate a three-dimensional cross-sectional image of a region of interest in the subject;
generating a combined image using the two-dimensional projection image and the three-dimensional cross-sectional image;
controlling selective generation of the two-dimensional projection image, the three-dimensional cross-sectional image, the combined image, or combinations thereof, based on one or more imaging specifications; and
a display device operatively coupled to the computing device and configured to display one or more of the two-dimensional projection image, the three-dimensional cross-sectional image, the combined image, or combinations thereof, based on the one or more imaging specifications.

2. The system of claim 1, wherein the computing device is configured to perform functional analysis of the subject based on the two-dimensional projection image, the three-dimensional cross-sectional image, the combined image, or combinations thereof.

3. The system of claim 1, wherein the one or more imaging specifications comprise real-time user input, protocol-based imaging specifications, or a combination thereof, for controlling one or more of a gantry position, a table position, x-ray exposure, one or more operating parameters corresponding to an x-ray source and one or more operating parameters corresponding to the display device.

4. The system of claim 1, wherein the large area detector comprises a plurality of detector cells, and wherein one or more of the plurality of detector cells are energy integrating detector cells, energy discriminating detector cells, or a combination thereof.

5. The system of claim 1, wherein the large area detector comprises a plurality of detector cells, and wherein the plurality of detector cells comprises a first set of detector cells having a first-resolution and a second set of detector cells having a second resolution, wherein the second resolution is less than the first resolution.

6. The system of claim 1, wherein a region in the large area detector comprises at least one of a flat-panel detector, a polygonal-shaped detector, a square detector, a rectangular detector, and a curved detector.

7. The system of claim 1, wherein the computing device is configured to use one or more of the two-dimensional projection image, the three-dimensional cross-sectional image, or a combination thereof to visualize one or more of the region of interest, an interventional device, or a movement of the interventional device within body of the subject in real-time during an interventional procedure.

8. The system of claim 1, wherein the computing device is configured to use the three-dimensional cross-sectional image to facilitate planning of an interventional procedure, conducting an interventional procedure, validating the efficacy of the interventional procedure, or a combination thereof.

9. The system of claim 1, wherein the integral CT and interventional system is configured to use the two-dimensional projection image to facilitate one or more of diagnostic evaluation, therapeutic intervention, or a combination thereof to the subject in the region of interest.

10. The system of claim 1, wherein the computing device is configured to perform one or more of determining a position of an interventional device inside body of the subject, detecting structural information of the region of interest, detecting functional information of the region of interest, facilitating a diagnostic evaluation, facilitating a therapeutic intervention, determining the efficacy of a therapeutic intervention provided by the interventional device, or combinations thereof, using the one of more of the two-dimensional projection image, the three-dimensional cross-sectional image, the combined image, functional analysis, or combinations thereof.

11. The system of claim 1, wherein the computing device is configured to adjust one or more of a position of a table, an angular orientation of a gantry, position of a gantry, or combinations thereof, corresponding to the integral computed tomography and interventional system based on one or more of a determined position of an interventional device inside body of the subject, protocol-based specification, and user input.

12. The system of claim 1, wherein the display device is configured to display one or more of images, processed information, and additional data received from an auxiliary system communicatively coupled to the display device, the integral computed tomography and interventional system, the computing device, or combinations thereof.

13. The system of claim 12, wherein the auxiliary system comprises one or more of an electrocardiogram monitor, an intravascular ultrasound system, an optical coherence tomographic system and a magnetic resonance system.

14. An imaging method, comprising:
acquiring projection data corresponding to a region of interest from one or more view angles using a large-area detector in an integral computed tomography and interventional imaging system;
processing the projection data to generate one or more of a two-dimensional projection image, a three-dimensional cross-sectional image of the region of interest, a combined image generated using the two-dimensional projection image and the three-dimensional cross-sectional image, or combinations thereof; and
selectively displaying the two-dimensional projection image, the three-dimensional cross-sectional image, the combined image, or combinations thereof, in real-time based on one or more imaging specifications.

15. The method of of claim 14, further comprising acquiring projection data over a sufficient temporal acquisition window.

16. The method of of claim 14, further comprising performing functional analysis of the region of interest based on the two-dimensional projection image, the three-dimensional cross-sectional image, the combined image, or combinations thereof.

17. The method of of claim 16, further comprising detecting one or more of structural information of the region of interest, determining functional information of the region of interest, detecting efficacy of a therapeutic interventional procedure, or combinations thereof, using one or more of the two-dimensional projection image, the three-dimensional cross-sectional image, the combined image, the functional analysis, or combinations thereof.

18. The method of claim 16, further comprising estimating an abnormality in cardiac wall motion, detecting ischemia, assessing perfusion, or combinations thereof, using one or more of the two-dimensional projection image, the three-dimensional cross-sectional image, the combined image, the functional analysis, or combinations thereof.

19. The method of claim 14, further comprising:
guiding movement of an interventional device in the vasculature of the patient to and from the region of interest using the the two-dimensional projection image, the three-dimensional cross-sectional image, the combined image, or combinations thereof; and
performing an interventional procedure at the region of interest using one or more of the two-dimensional projection image, the three-dimensional cross-sectional image, the combined image, or combinations thereof.

20. The method of of claim 14, further comprising registering the two-dimensional projection image with a corresponding three-dimensional cross-sectional image in a common coordinate system using a determined transformation that aligns the two-dimensional projection image with the corresponding three-dimensional cross-sectional image based on one or more designated fiducials.

21. The method of of claim 14, further comprising registering the two-dimensional projection image with corresponding three-dimensional cross-sectional image in a common coordinate system based on a known position of a gantry corresponding to the integral computed tomography and interventional imaging system at a time of acquisition of the projection data.

22. A non-transitory computer readable medium that stores instructions executable by one or more processors to perform a method for imaging, comprising:
acquiring projection data corresponding to a region of interest from one or more view angles using a large-area detector in an integral computed tomography and interventional imaging system;
processing the projection data to generate one or more of a two-dimensional projection image, a three-dimensional cross-sectional image of the region of interest, a combined image generated using the two-dimensional projection image and the three-dimensional cross-sectional image, or combinations thereof; and
selectively displaying the two-dimensional projection image, the three-dimensional cross-sectional image, the combined image, or combinations thereof, in real-time based on one or more imaging specifications.
